(19) United States
(12) Patent Application Publication
(10) Pub. No.: US 2025/0241675 A1
(43) Pub. Date: Jul. 31, 2025

(54) MEDICAL SYSTEMS, DEVICES, AND METHODS FOR PROVIDING TRACTION TO TISSUE

(71) Applicant: Boston Scientific Scimed, Inc., Maple Grove, MN (US)

(72) Inventors: Austin Grant JOHNSON, Worcester, MA (US); Barry WEITZNER, Acton, MA (US); Ryan V. WALES, Northborough, MA (US)

(73) Assignee: Boston Scientific Scimed, Inc., Maple Grove, MN (US)

(21) Appl. No.: 19/039,063

(22) Filed: Jan. 28, 2025

Related U.S. Application Data
(60) Provisional application No. 63/626,153, filed on Jan. 29, 2024.

(51) Int. Cl. A61B 17/3209; A61B 17/04

(52) U.S. Cl. CPC A61B 17/3209; A61B 17/0487; A61B 2017/32096

(57) ABSTRACT

A medical system may comprise: a sheath having a lumen; a medical instrument including a shaft and a clip; and a tether coupling the sheath to the clip. In a first configuration of the medical system, the shaft may extend through the lumen, and the clip may be coupled to the shaft. In a second configuration of the medical system, the clip may be separated from the shaft and the tether may couple the sheath to the clip.

### CROSS-REFERENCE TO RELATED APPLICATIONS

This application claims the benefit of priority to U.S. Provisional Application No. 63/626,153, filed on Jan. 29, 2024, which is incorporated by reference herein in its entirety.

### TECHNICAL FIELD

Various aspects of this disclosure relate generally to medical systems, devices, and methods for providing traction to tissue. In particular, aspects of the disclosure relate to systems, devices, and methods for clipping tissue in order to apply traction to the tissue and facilitate resection of the tissue.

### BACKGROUND

Endoscopic procedures may involve removing a lesion from a body lumen. For example, endoscopic submucosal dissection (ESD) utilizes endoscopic tools to remove cancerous or other lesions from the gastrointestinal tract. An instrument, such as an electrosurgical knife, may be passed through a working channel of an endoscope in order to cut and remove the lesion. In some procedures, a lifting agent may be injected under the lesion prior to removing the lesion, so as to facilitate access by the electrosurgical knife or other instrument. A need exists for applying traction or tension to a lesion or other portion of a body lumen to facilitate medical procedures, such as ESD.

### SUMMARY

Each of the aspects disclosed herein may include one or more of the features described in connection with any of the other disclosed aspects. Aspects of the disclosure may relate to systems, devices, and methods for applying traction to a tissue. The aspects disclosed herein may include a sheath tethered to a clip, which may be used in conjunction with an endoscope or other type of medical device to help apply traction to the tissue.

In an example, a medical system may comprise: a sheath having a lumen; a medical instrument including a shaft and a clip; and a tether coupling the sheath to the clip. In a first configuration of the medical system, the shaft may extend through the lumen, and the clip may be coupled to the shaft. In a second configuration of the medical system, the clip may be separated from the shaft and the tether may couple the sheath to the clip.

Any of the systems or devices disclosed herein may have any of the following features, alone or in any combination or subcombination. In the second configuration, the shaft may have been removed from the lumen. The sheath may include at least one opening. The clip may include at least one opening. The tether may extend through the at least one opening of the sheath and through the at least one opening of the clip. A capsule of the clip may include the at least one opening of the clip. The medical instrument may be a first medical instrument. In the second configuration, a second medical instrument may extend through the sheath. A distal portion of the sheath may have a straight shape in the first configuration and a bowed shape in the second configuration. The distal portion may curve away from a central longitudinal axis of a more proximal portion of the sheath. The sheath may include a braid, a coil, or a shape-memory material. The medical system may comprise an endoscope. The sheath may be external to the endoscope. The medical instrument may be a first medical instrument. The medical system may further include a second medical instrument extending through a working channel of the endoscope. The second medical instrument may be configured to grasp the tether. The medical system may further include a fastener that couples a shaft of the endoscope to the sheath. The fastener may be configured such that the sheath is axially and rotatably movable relative to the shaft of the endoscope. The medical instrument may be a first medical instrument. In the second configuration of the medical system, a second medical instrument may be configured to resect tissue and extend through a working channel of the endoscope. The sheath, the tether, and the clip may be configured to apply traction to a target tissue. A proximal end of the sheath may include a feature configured to be gripped by a user.

In another example, a medical system may comprise an endoscope having a shaft and a sheath external to the shaft. The sheath may have a lumen. The medical system may also comprise a clip tethered to a distal end of the sheath, such that in a configuration in which the clip is deployed to a target tissue, the clip is coupled to the sheath. The sheath and the clip may be configured to apply traction to the target tissue.

The systems and devices disclosed herein may have any of the following features, alone or in any combination or subcombination. The sheath may be biased toward a configuration in which a distal end of the sheath is bowed.

In an example, a medical method may comprise: inserting an endoscope coupled to an external sheath into a body lumen of a subject; advancing the endoscope and the external sheath through the body lumen; and using a first medical instrument extending through a lumen of the external sheath to deploy a clip to a target tissue. The deployed clip may be coupled to the external sheath. The method may further include using a second medical instrument extending through a working channel of the endoscope to treat a lesion of the body lumen.

Any of the methods disclosed herein may include any of the following features, alone or in any combination or subcombination. Advancing the endoscope and the external sheath may include using a third medical instrument to grasp the sheath or a tether coupling the clip to the sheath. The method may further include, after deploying the clip to the target tissue, advancing a third medical instrument through the lumen of the sheath to grasp the lesion.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIG. 1 depicts an exemplary system for applying traction to a tissue.

FIG. 2 depicts a distal portion of the exemplary system of FIG. 1 in an exemplary use.

FIG. 3 depicts the distal portion of the exemplary system of FIG. 1 in an alternative exemplary use.

FIGS. 4A and 4B depict an exemplary sheath that may be used along with or as part of the system of FIG. 1.

FIG. 5 depicts aspects of another exemplary system for applying traction to a tissue.

### DETAILED DESCRIPTION

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values+/−10% of a stated value. Unless otherwise stated, ranges disclosed herein include the end points of the ranges. The use of ordinals (e.g., first, second, third, fourth) herein is for convenient reference to different examples and does not imply any ordered or other type of relationship among identified elements. The ordinal applied to a given element is arbitrary, and any alternative ordinal may be used with the element.

A system for applying traction to tissue may include an endoscope and a sheath that is external to the endoscope. The sheath may receive a medical clip device. A deployable portion of the medical clip may be tethered to the sheath. Deployment of the clip to tissue may help to apply traction to the tissue because the clip remains tethered to the sheath. Instruments may be delivered by the endoscope in order to perform one or more procedures (e.g., ESD). For example, the clip may apply tension to a lesion, and an electrosurgical knife or other instrument may cut the lesion. In other examples, the clip may anchor the sheath in a desired location, such that a medical instrument that is passed through the sheath may grasp a lesion. An instrument (e.g., electrosurgical knife) delivered by the endoscope may cut the lesion.

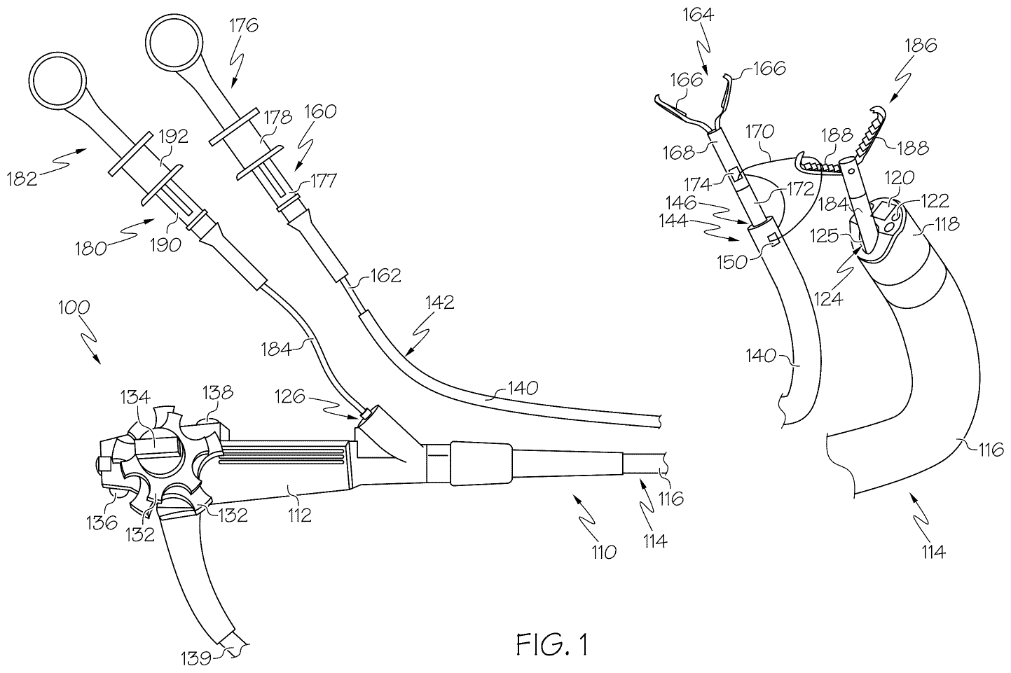

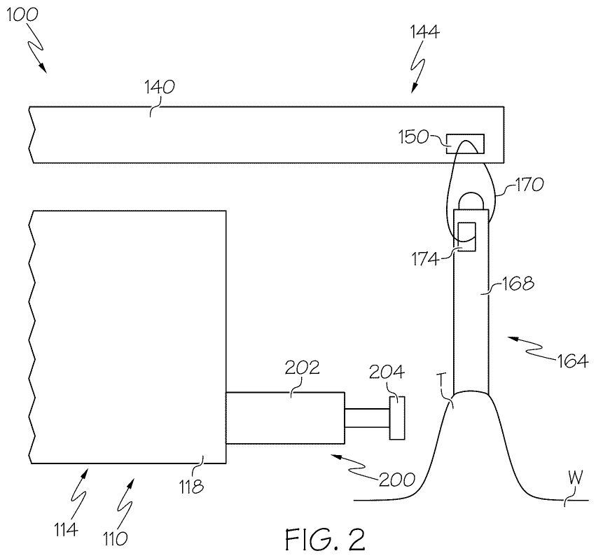

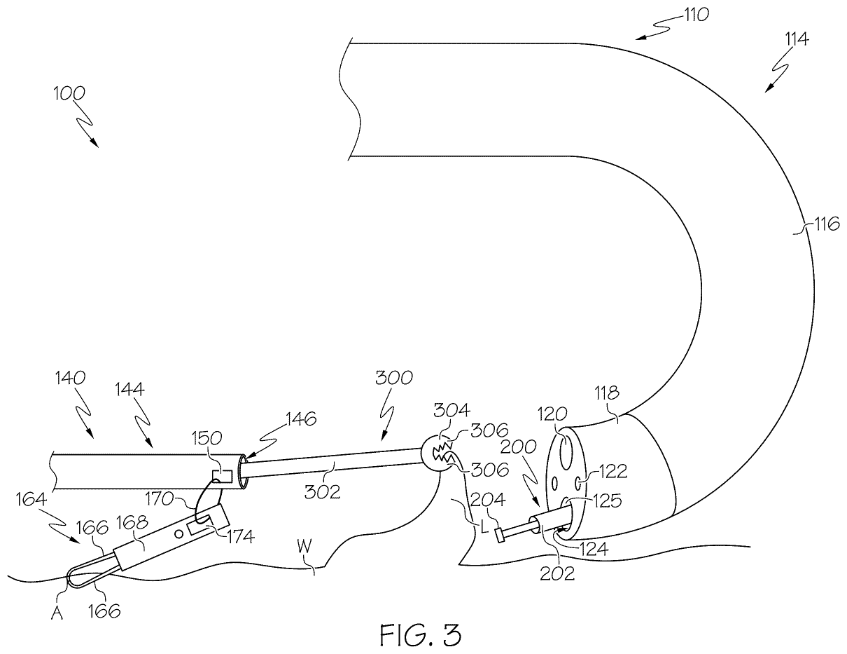

FIG. 1 depicts an exemplary medical system 100. Medical system 100 may include an endoscope 110 and a sheath 140 (e.g., a catheter). A first medical instrument 160 may extend through sheath 140. As described herein, first medical instrument 160 may apply traction and/or tension to a target tissue (e.g., to a lesion or to an area neighboring a lesion) and/or may create a tissue flap. A second medical instrument 180 may extend through a working channel of endoscope 110 to facilitate delivery of first medical instrument 160. As described in further detail below, with respect to FIGS. 2 and 3, additional medical instruments may extend through sheath 140 and/or endoscope 110 in order to facilitate resection of the lesion.

Endoscope 110 may have any of the features of any endoscope known in the art. For example, endoscope 110 may include a handle 112 and an insertion portion 114. Insertion portion 114 may have a shaft 116 and a distal tip 118. Distal tip 118 may include one or more imaging devices 120 and/or lighting elements 122. Imaging device 120 may include a camera and/or other optical device(s) for transmitting an image from a body lumen to a user (e.g., optical fiber, lens, image sensor, etc.). Lighting elements 122 may include any element that may deliver light to a procedure site, such as light emitting diode(s), optical fibers, or other types of light sources. Distal tip 118 may also include a distal opening 125 of a working channel 124. Working channel 124 may extend from the handle, through the shaft, to distal opening 125.

Handle 112 may include a port 126 that defines a proximal opening of working channel 124. An instrument may be inserted into port 126, through working channel 124, and out of distal opening 125 of working channel 124. Thus, an instrument may be deployed at a procedure site within a body lumen.

Handle 112 may include one or more control elements for controlling aspects of insertion portion 114. For example, handle 112 may include one or more steering actuators 132 (e.g., knobs or levers) and/or one or more braking actuators 134 (e.g., knobs or levers). Steering actuators 132 may steer a distal portion of shaft 116 (e.g., by manipulating an articulation joint of shaft 116). Braking actuators 134 may help to inhibit steering of the distal portion of shaft 116. Handle 112 may also include one or more actuators 136 for controlling electronic elements of distal tip 118. For example, actuator(s) 136 may control image capture, lighting levels, or other aspects of imaging device 120 and/or lighting elements 122. Handle 112 may also include one or more valves 138 for delivering air, water, etc. to distal tip 118 and/or applying suction or negative pressure to distal tip 118.

An umbilicus 139 may extend proximally from handle 112. Umbilicus 139 may be used to couple endoscope 110 to capital equipment, such as electronic controller(s), and/or source(s) of air, water, and/or suction.

Although endoscopes are particularly referenced herein, the disclosure also encompasses other types of devices, such as duodenoscopes, bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to a herein should be understood to encompass any of the above medical devices.

Sheath 140 may extend from a proximal end 142 to a distal end 144. Sheath 140 may be external to endoscope 110, such that sheath 140 may be external to, and extend alongside of, an outer surface of shaft 116. Sheath 140 may have a tubular shape and may be flexible. For example, sheath 140 may be formed from a polymer or polymer-like material (e.g., via extrusion). Sheath 140 may define a lumen 146 that extends from proximal end 142 to distal end 144. Lumen 146 may have open proximal and distal ends, such that a medical instrument may be passed from proximal end 142 to distal end 144 via lumen 146. A diameter of lumen 146 may be sized so that a desired medical instrument may be passed through lumen 146. For example, lumen 146 may have a diameter of approximately 1.0 mm to approximately 4.0 mm or approximately 2.0 mm to approximately 3.0 mm. Proximal end 142 may include one or more features (e.g., ridges, roughened portion(s), widened portion, etc.) to help a user to grip proximal end 142.

Sheath 140 may be packaged with first medical instrument 160 in a first configuration, with first medical instrument 160 extending through lumen 146. First medical instrument 160 may be or may include, for example, a medical clip. First medical instrument 160 may have any of the features of any medical clip device known in the art. First medical instrument 160 may include a shaft 162 and a clip 164. Clip 164 may include a pair of clip arms 166, which may be partially contained within a capsule 168. Clip 164 may be removably coupled to shaft 162 via a bushing 172.

A handle 176 may be disposed at a proximal end of shaft 162. Handle 176 may include a handle body 177 and one or more actuators 178 for controlling aspects of clip 164. For example, actuator 178 may be a spool that moves longitudinally along handle body 177. Handle 176 may have any of the features of any medical device handle known in the art. Handle 176 may include additional and/or alternative actuators. A configuration of handle 176 is merely exemplary and shown for illustrative purposes. An operation of first medical instrument 160 and further features of first medical instrument 160 are described below.

Sheath 140 and clip 164 (e.g., capsule 168 of clip 164) may be coupled (fixed) to one another via a tether 170. For example, tether 170 may be a string, wire, suture, thread, cable, band, or other member. Distal end 144 of sheath 140 may include one or more openings 150 (at least one opening 150) formed in a wall of sheath 140. Openings 150 may be formed near a distalmost end of sheath 140. For example, one or more openings 150 may be formed in a radially outer wall of sheath 140. For example, sheath 140 may include two or more (e.g., exactly two) openings 150. When viewing sheath 140 from the side, two openings 150 of sheath 140 may be aligned with one another, as shown in FIG. 1, such that one of the openings 150 is not visible in FIG. 1. Although one or more openings 150 are shown as being rectangular, one or more openings 150 may have any suitable shape (e.g., circular, ovular, square, slit, etc.).

A portion of clip 164, such as capsule 168, may include one or more openings 174 (at least one opening 174). Openings 174 may have any of the features of openings 150. Openings 174 may be formed in a radially outer surface of capsule 168. In some examples, clip 164 may include two or more (e.g., exactly two) openings 174. When viewing clip 164 from the side, two openings 174 may be aligned with one another, as shown in FIG. 1, such that only one side opening 174 is visible in FIG. 1. Although one or more openings 174 are shown as being rectangular, one or more openings 174 may have any suitable shape (e.g., circular, ovular, square, slit, etc.).

Tether 170 may extend through openings 150 and 174 to couple sheath 140 to clip 164. For example, tether 170 may extend through both of two openings 150 and both of two openings 174. Tether 170 may have a length such that, after clip 164 is deployed to a tissue (separated from shaft 162), a force from sheath 140 on clip 164 via tether 170 may exert traction on the tissue, as described in further detail below.

Second medical instrument 180 may extend through working channel 124 of insertion portion 114. For example, second medical instrument 180 may extend through port 126 and extended out of distal opening 125 of working channel 124. Second medical instrument 180 may include a handle 182, a shaft 184 extending distally from handle 182, and an end effector 186. End effector 186 may be a forceps, grasper, or other type of medical device having jaws 188 (e.g., a pair of jaws 188).

Handle 182 may have any of the features of handle 176. Handle 182 may include a handle body 190 and an actuator 192. Actuator 192 may be, for example, a spool that is longitudinally movable along handle body 190. Actuator 192 may control end effector 186 and/or shaft 184. For example, actuator 192 may control opening and/or closing of jaws 188 and/or steering of shaft 184. Handle 182 may include additional and/or alternative actuators. A configuration of handle 182 is merely exemplary and shown for illustrative purposes.

A method of using system 100 in the configuration as shown in FIG. 1 will now be described. Prior to insertion of insertion portion 114 into a body lumen of a subject (e.g., via an orifice such as a mouth or anus), second medical instrument 180 may be inserted into port 126 and extended distally through working channel 124 until end effector 186 extends distally from distal opening 125. Jaws 188 may be used to grip or grasp tether 170. Although jaws 188 are shown as being open or partially open in FIG. 1, it will be appreciated that jaws 188 may be closed while grasping tether 170.

With jaws 188 grasping tether 170, endoscope 110 and sheath 140 may be inserted into a bodily orifice of a subject. As endoscope 110 is advanced through a body lumen of the subject, jaws 188 may pull tether 170 and, therefore, a distal portion of sheath 140, distally as endoscope 110 moves distally. In other words, endoscope 110 may drag sheath 140 along with it, because jaws 188 grip tether 170. Thus, second medical instrument 180 may facilitate insertion of system 100, including endoscope 110 and sheath 140 into a body lumen of the subject and advancement of the system 100 through the body lumen to a target site. Shaft 116 of endoscope 110 may be actively steered using steering actuators 132, and distal end 144 of sheath 140 and clip 164 may be passively steered by being pulled along by medical instrument 180. Alternatively, jaws 188 may grip distal end 144 of sheath 140 itself, rather than tether 170, in order to position sheath 140.

Once clip 164/sheath 140 is positioned at a target site, a user may deploy clip 164 to the target site, thereby separating clip 164 from shaft 162. In some examples, the user may deploy clip 164 while jaws 188 continue to grip tether 170. Alternatively, the user may disengage jaws 188 from tether 170 (e.g., by utilizing actuator 192 to open jaws 188 and by moving an entirety of handle 182 to move end effector 186 proximally) before the user deploys clip 164. Clip 164 may be deployed using any method/approach that is typically used to deploy clip 164. For example, clip 164 may be advanced distally by advancing handle 176 distally, and clip 164 may be deployed using actuator 178. As clip 164 is deployed, clip arms 166 may close, and capsule 168 may separate from bushing 172. Thus, clip 164 may be separated/severed from shaft 162 and bushing 172. However, the mechanics described above are merely exemplary, and first medical instrument 160 may be operated using any mechanisms known in the art. Clip 164 may remain tethered to sheath 140 by tether 170.

FIG. 2 shows a distal portion of system 100 in a first exemplary use after clip 164 has been deployed to a target tissue T. In FIG. 2, clip arms 166 are not visible because they are embedded within target tissue T. The particular manner in which clip arms 166 are deployed to and grip target tissue T is not limited by this disclosure. As shown in FIG. 2, tether 170 couples capsule 168 to sheath 140 after clip 164 has been deployed. In FIG. 2, second medical instrument 180 has been removed from endoscope 110, such that sheath 140 is no longer tethered to endoscope 110 by second medical instrument 180.

Clip 164 may apply traction to target tissue T due to a coupling of clip 164 to sheath 140 by tether 170. Sheath 140 may be positioned so that it is spaced apart from target tissue T and tether 170 is taut. In some examples, clip 164 may be deployed to target tissue T with jaws 188 of second medical instrument 180 still grasping tether 170. After clip 164 is positioned, jaws 188 may be utilized to position sheath 140 in a desired location spaced apart from target tissue T, before second medical instrument 180 is removed from working channel 124. In aspects, a grasper, snare, basket, or like device may be used to position sheath 140. If a snare or basket is used, a feature on sheath 140 may provide a location for the snare or basket to fall into while closing.

In the configuration of FIG. 2, shaft 162 of first medical instrument 160 may have been removed from lumen 146 of sheath 140, such that sheath 140 is in a second (empty) configuration. Alternatively, shaft 162 may remain within lumen 146 after clip 164 is deployed (separated from shaft 162).

Because sheath 140 is spaced apart from target tissue T and tether 170 is taut, sheath 140 and tether 170 may pull target tissue T away from surrounding portions of a body lumen wall W. In the view of FIG. 2, target tissue T may be pulled upward, away from body lumen wall W. Tether 170 may exert a force on target tissue T at a non-zero angle relative to surrounding portions of body lumen wall W. For example, tether 170 may exert a force on tissue T at an angle of at least approximately 30 degrees, at least approximately 45 degrees, at least approximately 60 degrees, at least approximately 75 degrees, or approximately 90 degrees. The angles discussed above may be a smallest angle that is drawn between a direction of a force on target tissue T and a surrounding body lumen wall W. A force exerted by sheath 140/tether 170/clip 164 on target tissue T may be sufficient so as to pull target tissue T away from surrounding portions of body lumen wall W, which may help to allow resection or other manipulation (treatment) of target tissue T.

As shown in FIG. 2, a third medical instrument 200 may be extended distally through working channel 124 (see FIG. 1) and out of distal opening 125 (also shown in FIG. 1). Third medical instrument 200 may include a shaft 202 and a distal tool 204. In some examples, third medical instrument 200 may be an electrosurgical knife, and distal tool 204 may be electrified and/or may inject a fluid (e.g., via an internal lumen). However, third medical instrument 200 may be any other suitable type of instrument, such as another type of knife, a snare, or a needle. Third medical instrument 200 may be utilized (e.g., via a handle having any of the features of handles 176, 182) in order to cut, resect, or otherwise treat target tissue T, for example, to separate tissue T from body lumen wall W.

Withdrawing sheath 140 proximally until sheath 140 is removed from the body lumen may remove target tissue T (gripped by clip 164) from a subject's body. Alternatively, other instrument(s) (not shown) may be extended through working channel 124 to capture target tissue T and/or to sever tether 170. In such examples, the other instrument (e.g., basket, snare, grasper, etc.) may be used to remove target tissue T from the subject's body.

FIG. 3 shows a distal portion of system 100 in a second exemplary use after clip 164 has been deployed to a target tissue A. Whereas, in the first exemplary use of FIG. 2, clip 164 has been deployed to target tissue T that is a lesion to be resected, in the second exemplary use of FIG. 2, clip 164 is deployed to target tissue A, which is not a lesion L to be resected. As described in further detail below, in the second exemplary use of FIG. 2, the clip 164 may anchor sheath 140 to a desired location. A fourth medical instrument 300 may be extended through sheath 140 to apply traction to lesion L to facilitate resection of lesion L.

The particular manner in which clip arms 166 are deployed to and grip target tissue A is not limited by this disclosure. As shown in FIG. 2, tether 170 couples capsule 168 to sheath 140 after clip 164 has been deployed. In FIG. 3, second medical instrument 180 has been removed from endoscope 110, such that sheath 140 is no longer tethered to endoscope 110 by second medical instrument 180.

Because clip 164 is connected to (tethered to) sheath 140 by tether 170, clip 164 may help anchor distal end 144 of sheath 140 in a desired location. For example, distal end 144 may be anchored near a lesion L that is desired to be resected. In the example of FIG. 3, distal end 144 may be near to, for example, proximal of, lesion L.

A user may insert fourth medical instrument 300 into lumen 146 (which has been vacated by second medical instrument 180) and may extend fourth medical instrument 300 distally from a distal opening of lumen 146. In some examples, fourth medical instrument 300 may include a shaft 302 and an end effector 304. As shown in FIG. 3, end effector 304 may be a grasper or forceps having two jaws 306.

Prior to, after, or concurrently with inserting fourth medical instrument 300 into lumen 146, the user may adjust a position of endoscope 110, such that endoscope 110 (e.g., imaging device 120 of endoscope 110) may face lesion L and a distalmost end of sheath 140/end effector 304. For example, as shown in FIG. 3, endoscope 110 may be in a retroflexed position, such that imaging device 120 faces proximally.

With or without visual assistance from imaging device 120, the user may use end effector 304 to grasp and apply traction to lesion L. End effector 304 may pull lesion L away from a surrounding (e.g., adjacent) tissue wall W of the body lumen. The traction applied to lesion L may facilitate resection of lesion L.

As discussed above with respect to FIG. 2, third medical instrument 200 may be extended distally through working channel 124 and out of distal opening 125 and may be used to resect and/or remove lesion L from the body lumen. In some examples, fourth medical instrument 300 may continue to grip lesion L, and sheath 140, along with fourth medical instrument 300, may be withdrawn from the body. In other examples, as described above, an instrument may be extended from working channel 124 in order to remove/resect lesion L. In some examples, tether 170 may be severed by third medical instrument 200 or by another mechanism (e.g., by a feature of sheath 140). In some examples, distal end 144 may be separated from other portions of sheath 140 so that sheath 140 may be removed from the body lumen.

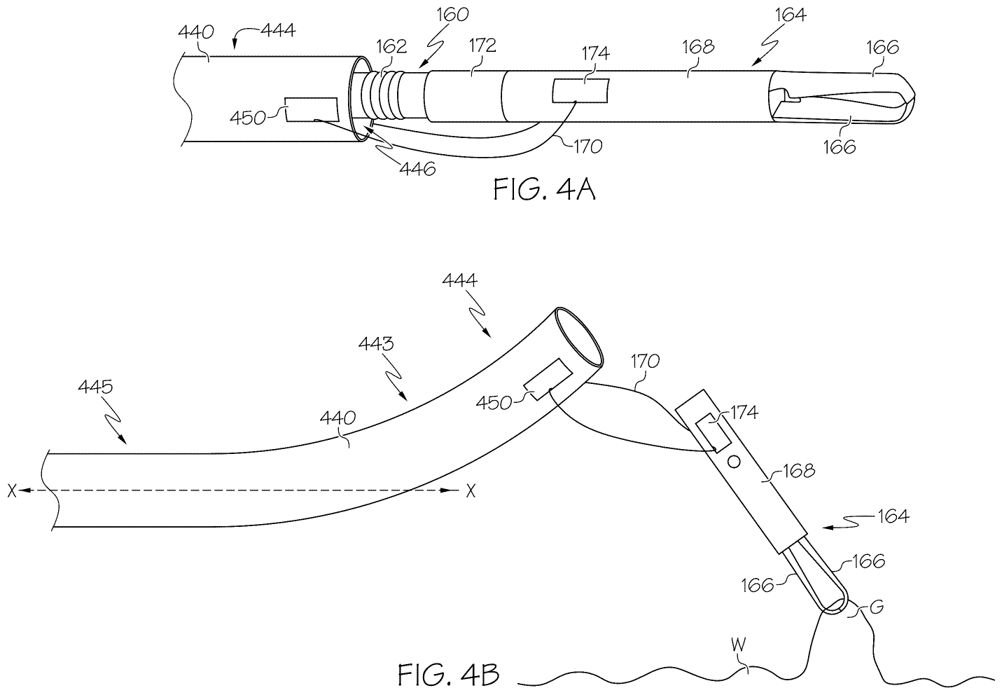

FIGS. 4A and 4B show an alternative sheath 440 that may be used in place of sheath 140 in system 100. Unless otherwise specified, sheath 440 may have any of the properties of sheath 140. Sheath 440 may be used in either of the methods of FIGS. 2 and/or 3. Sheath 440 may have a proximal end (not shown but having any of the features of proximal end 142) and a distal end 444. Sheath 440 may have one or more openings 450, having any of the properties of openings 150. Tether 170 may extend through opening(s) 450.

FIG. 4A shows sheath 440 with shaft 162 of first medical instrument 160 extending through a lumen 446. FIG. 4B shows sheath 440 after clip 164 has been deployed and shaft 162 has been removed from lumen 446. Sheath 440 may have a straightened configuration in FIG. 4A and a relaxed (natural) configuration in FIG. 4B. A distal portion 443 (which includes distal end 444) of sheath 440 may include shape-memory material or other properties such that, in the relaxed configuration (FIG. 4B), distal portion 443 may be curved (e.g., arcuate) relative to a more proximal region 445 of sheath 440. In other words, in the relaxed configuration, distal portion 443 may be biased to bow outward relative to a central longitudinal axis X of more proximal region 445. In other words, distal portion 443 may be biased to curve away from central longitudinal axis X. For example, distal portion 443 may be biased to a bowed configuration. For example, sheath 440 or a portion of sheath 440 may include a composite braid or coil that is manufactured (e.g., braided or wound) so that distal portion 443 of sheath bows outward in a relaxed configuration. In alternatives, sheath 440 may include a slotted hypotube made from stainless steel or Nitinol (nickel titanium alloy).

As sheath 440 is positioned in a body lumen, shaft 162 may be sufficiently rigid so as to maintain shaft 162 in a straightened configuration, with distal portion 443 of sheath 440 having a straight shape. The straightened configuration of shaft 162 may make placement of distal end 444 of sheath 440 easier and/or may decrease a risk of distal end 444 getting caught during navigation/advancement of sheath 440.

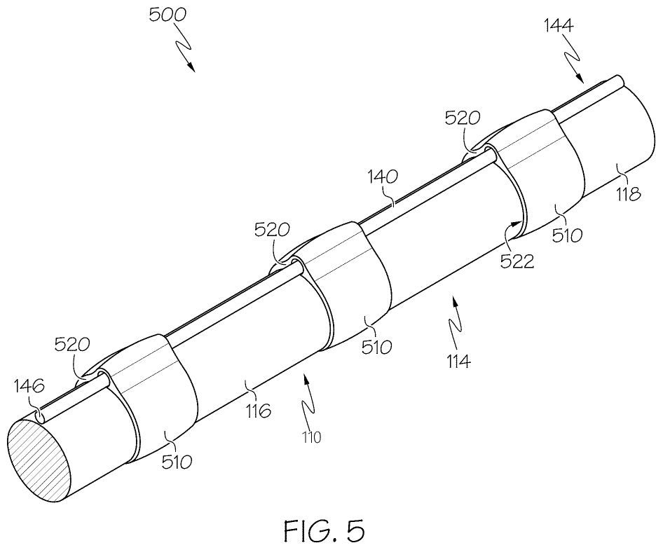

The curved shape of distal portion 443 may help sheath 440, tether 170, and clip 164 to apply traction to target tissue G (which may be a target tissue T that is a lesion or a target tissue A that is an anchoring location). Sheath 440 may be positioned (using endoscope 110 as described above, or below with respect to FIG. 5) and clip 164 may be deployed to target tissue G, as discussed above for FIGS. 1-3. After clip 164 is deployed, shaft 162 may be removed proximally from lumen 446, which may cause distal portion 443 to bow outward (e.g., toward a bowed configuration) and help to apply traction to target tissue G. A bowed shape of distal portion 443 may create additional distance between distal end 444 and wall W of a body lumen, creating additional leverage on tissue G. Furthermore, a shape of sheath 440 in the relaxed shape may provide additional control over the traction applied, including a direction of the traction. In some examples, sheath 440 may have sufficient torsional stiffness, such that sheath 440 may be rotated about longitudinal axis X. Such rotation may provide additional control over a direction and/or magnitude of traction applied to target tissue G. As discussed above with respect to FIGS. 2 and 3, the traction applied by sheath 440, tether 170, and clip 164 may facilitate resection of a lesion.

FIG. 5 depicts a cross-sectional view of a portion of an alternative system 500 using endoscope 110 and sheath 140. System 500 may have any property of system 100, except as described herein. Although shaft 116 of endoscope 110 is shown as having a solid cross-section for ease of illustration, it will be appreciated that endoscope 110 may have various internal lumens or other structures.

Whereas system 100 may utilize second medical instrument 180 and tether 170 to position sheath 140, system 500 may utilize one or more fasteners 510 to couple sheath 140 to shaft 116 of endoscope 110. For example, fasteners 510 may be any coupling mechanism, such as clips, bands, rings, loops, ties, or a combination thereof. Fasteners 510 may be rigid, flexible, or a combination of rigid and flexible. As shown in FIG. 5, fasteners 510 may each include a first lumen 520 and a second lumen 522. First lumen 520 may be sized so as to receive sheath 140, and second lumen 522 may be sized so as to receive shaft 116. In some examples, first lumen 520 may be in fluid communication with second lumen 522. A size of first lumen 520 may be large enough to allow axial and/or rotational movement of sheath 140 relative to endoscope 110, for example, to provide control over the applied traction. First lumen 520 may be small enough so as to retain sheath 140 alongside endoscope 110 during navigation of endoscope 110 to a procedure location in a body lumen.

System 100 may include any number of fasteners 510, which may have any suitable spacing along shaft 116/sheath 140. For example, fasteners 510 may be disposed only on a distal portion of shaft 116/sheath 140. Fasteners 510 may alternatively be spaced along an entire length of shaft 116/sheath 140. Fasteners 510 may be evenly or unevenly spaced. In some examples, system 100 may include only one fastener 510.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

We claim:

1. A medical system comprising:
a sheath having a lumen;
a medical instrument including a shaft and a clip; and
a tether coupling the sheath to the clip,
wherein, in a first configuration of the medical system, the shaft extends through the lumen, and the clip is coupled to the shaft, and
wherein, in a second configuration of the medical system, the clip is separated from the shaft and the tether couples the sheath to the clip.

2. The medical system of claim 1, wherein, in the second configuration, the shaft has been removed from the lumen.

3. The medical system of claim 1, wherein the sheath includes at least one opening, wherein the clip includes at least one opening, and wherein the tether extends through the at least one opening of the sheath and through the at least one opening of the clip.

4. The medical system of claim 3, wherein a capsule of the clip includes the at least one opening of the clip.

5. The medical system of claim 1, wherein the medical instrument is a first medical instrument, and wherein, in the second configuration, a second medical instrument extends through the sheath.

6. The medical system of claim 1, wherein a distal portion of the sheath has a straight shape in the first configuration and a bowed shape in the second configuration.

7. The medical system of claim 6, wherein the distal portion curves away from a central longitudinal axis of a more proximal portion of the sheath.

8. The medical system of claim 6, wherein the sheath includes a braid, a coil, or a shape-memory material.

9. The medical system of claim 1, wherein the medical system further comprises an endoscope, and wherein the sheath is external to the endoscope.

10. The medical system of claim 9, wherein the medical instrument is a first medical instrument, wherein the medical system further includes a second medical instrument extending through a working channel of the endoscope, and wherein the second medical instrument is configured to grasp the tether.

11. The medical system of claim 9, wherein the medical system further includes a fastener that couples a shaft of the endoscope to the sheath.

12. The medical system of claim 11, wherein the fastener is configured such that the sheath is axially and rotatably movable relative to the shaft of the endoscope.

13. The medical system of claim 9, wherein the medical instrument is a first medical instrument, and wherein, in the second configuration of the medical system, a second medical instrument configured to resect tissue extends through a working channel of the endoscope.

14. The medical system of claim 1, wherein the sheath, the tether, and the clip are configured to apply traction to a target tissue.

15. The medical system of claim 1, wherein a proximal end of the sheath includes a feature configured to be gripped by a user.

16. A medical system comprising:
an endoscope having a shaft;
a sheath external to the shaft, wherein the sheath has a lumen; and
a clip tethered to a distal end of the sheath, such that in a configuration in which the clip is deployed to a target tissue, the clip is coupled to the sheath,
wherein the sheath and the clip are configured to apply traction to the target tissue.

17. The medical system of claim 16, wherein the sheath is biased toward a configuration in which a distal end of the sheath is bowed.

18. A medical method comprising:
inserting an endoscope coupled to an external sheath into a body lumen of a subject;
advancing the endoscope and the external sheath through the body lumen;
using a first medical instrument extending through a lumen of the external sheath to deploy a clip to a target tissue, wherein the deployed clip is coupled to the external sheath; and
using a second medical instrument extending through a working channel of the endoscope to treat a lesion of the body lumen.

19. The medical method of claim 18, wherein advancing the endoscope and the external sheath includes using a third medical instrument to grasp the sheath or a tether coupling the clip to the sheath.

20. The medical method of claim 18, further comprising, after deploying the clip to the target tissue, advancing a third medical instrument through the lumen of the sheath to grasp the lesion.
